(19) United States
(12) Patent Application Publication
(10) Pub. No.: US 2021/0401807 A1
(43) Pub. Date: Dec. 30, 2021

(54) NEW COMPOSITION COMPRISING AMORPHOUS NANOPOROUS SILICA PARTICLES

(71) Applicant: VICORE PHARMA AB, Stockholm (SE)

(72) Inventors: Carl-Johan DALSGAARD, Stockholm (SE); Adam FEILER, Hässelby (SE); Chunfang ZHOU, Stockholm (SE); Paulina PASZKIEWICZ, Stockholm (SE); Xin XIA, Bandhagen (SE)

(21) Appl. No.: 17/292,226

(22) PCT Filed: Nov. 6, 2019
(86) PCT No.: PCT/GB2019/053137

§ 371 Date: May 7, 2021

(30) Foreign Application Priority Data
Nov. 7, 2018 (GB) 1818164.4

(51) Int. Cl. A61K 31/4178; A61K 9/50; A61K 9/00

(52) U.S. Cl. CPC A61K 31/4178; A61K 9/5089; A61K 9/0075; A61K 9/501

(57) ABSTRACT

According to the invention, there is provided a pharmaceutical composition suitable for administration to the lung, which composition comprises a plurality of amorphous nanoporous silica particles, in which N-butyloxycarbonyl-3-(4-imidazol-1-yl-methylphenyl)-5-iso-butylthiophene-2-sulfonamide or a pharmaceutically-acceptable salt thereof is loaded into the pores of said silica particles, and wherein the silica particles have: (a) a mass median aerodynamic diameter that is between about 0.1 μm and about 10 μm; and (b) a geometric standard deviation that is less than about 4. Such compositions find particular utility in the treatment of an interstitial lung diseases, such as idiopathic pulmonary fibrosis and sarcoidosis, which diseases may be treated with such compositions for example by way of pulmonary administration.

### FIELD OF THE INVENTION

This invention relates to new pharmaceutical compositions, their use as medicaments and particularly to their administration to the lung to treat, for example, interstitial lung diseases.

### BACKGROUND AND PRIOR ART

Interstitial lung diseases (ILDs) are a group of lung diseases that affect the interstitium, characterised by tissue around alveoli becoming scarred and/or thickened, and so inhibiting the respiratory process.

ILDs are distinct from obstructive airway diseases (e.g. chronic obstructive airway disease (COPD) and asthma), which are typically characterized by narrowing (obstruction) of bronchi and/or bronchioles. ILDs may be caused by injury to the lungs, which triggers an abnormal healing response but, in some cases, these diseases have no known cause. ILDs can be triggered by chemicals (silicosis, asbestosis, certain drugs), infection (e.g. pneumonia) or other diseases (e.g. rheumatoid arthritis, systemic sclerosis, myositis, hypersensitivity pneumonitis or systemic lupus erythematosus).

The most common ILDs are idiopathic pulmonary fibrosis (IPF) and sarcoidosis, both of which are characterised by chronic inflammation and reduced lung function.

Sarcoidosis is a disease of unknown cause that is characterised by collections of inflammatory cells that form lumps (granulomas), often beginning in the lungs (as well as the skin and/or lymph nodes, although any organ can be affected). When sarcoidosis affects the lungs, symptoms include coughing, wheezing, shortness of breath, and/or chest pain.

Treatments for sarcoidosis are patient-specific. In most cases, symptomatic treatment with non-steroidal anti-inflammatory drugs (NSAIDs) is possible, but for those presenting lung symptoms, glucocorticoids (e.g. prednisone or prednisolone), antimetabolites and/or monoclonal anti-tumor necrosis factor antibodies are often employed.

IPF on the other hand is a chronic lung disease characterized by a progressive and irreversible decline in lung function caused by scarring of the lungs. Symptoms typically include cough and shortness of breath. Although less prevalent than asthma and COPD, mortality rates from IPF are much higher (e.g. 5 times higher than that of asthma, despite asthma being 100 times more prevalent).

Current treatment of IPF includes oxygen supplementation. Medications that are used include pirfenidone or nintedanib, but with only limited success in slowing the progression of the disease. Further, both of these drugs commonly cause (predominantly gastrointestinal) side-effects.

IPF affects about 5 million people globally. Average life expectancy after diagnosis is around four years.

There are drawbacks associated with all of the aforementioned ILD drug treatments and there is a real clinical need for safer and/or more effective treatments.

International patent application WO 2016/139475 discloses the use of compounds that are angiotensin II (Ang II) receptor agonists, including selective agonists of the Ang II type 2 receptor (hereinafter the AT2 receptor), and in particular N-butyloxycarbonyl-3-(4-i midazol-1-ylmethylphenyl)-5-iso-butylthio-phene-2-sulfonamide (Compound 21 or in short C21), for the therapeutic treatment of IPF.

The Renin-Angiotensin System (RAS) is a key regulator of blood pressure homeostasis. Renin, a protease, cleaves its only known substrate (angiotensinogen) to form angiotensin I, which in turn serves as substrate to angiotensin converting enzyme (ACE) to form Ang II. The endogenous hormone Ang II is a linear octapeptide (Asp¹-Arg²-Val³-Tyr⁴-lle⁵-His⁶-Pro⁷-Phe⁸), and is an active component of the RAS.

The AT1 receptor is expressed in most organs, and is believed to be responsible for the majority of the pathological effects of Ang II. The safety and efficacy of losartan (an AT1-receptor inhibitor) are currently being investigated in a phase II open-label clinical trial of IPF (www.clinicaltrials.gov identifier NCT00879879).

Several studies in adult individuals appear to demonstrate that, in the modulation of the response following Ang II stimulation, activation of the AT2 receptor has opposing effects to those mediated by the AT1 receptor.

The AT2 receptor has also been shown to be involved in apoptosis and inhibition of cell proliferation (de Gasparo M et al, Pharmacol. Rev., 2000; 52:415-472).

AT2 receptor agonists have also been shown to be of potential utility in the treatment and/or prophylaxis of disorders of the alimentary tract, such as dyspepsia and irritable bowel syndrome, as well as multiple organ failure (see international patent application WO 99/43339).

The expected pharmacological effects of agonism of the AT2 receptor are described in general in de Gasparo M et al., 2000. It is not mentioned that agonism of the AT2 receptor may be used to treat IPF.

Formulative work carried out in respect of C21 and salts thereof has proven extremely difficult. Attempts to provide stable solid state formulations have produced blends with conventional excipients that are chemically unstable. As a consequence, C21 is presently formulated as a solution for oral dosing.

Particles comprising nanoporous (mesoporous) silica materials have been disclosed for use in general pharmaceutical and cosmetic applications in inter alia international patent application WO 2012/035074. Here, poorly soluble active ingredients are incorporated within nanopore channels of the silica particles. The use of similar particles with a specific particle size distribution for delivery of active ingredients to the respiratory tract are disclosed in international patent application WO 2018/202818.

The use of porous materials for potential delivery of active ingredients to the lung has been disclosed. For example, U.S. Pat. Nos. 6,254,854, 6,740,310 and 7,435,408 all disclose polymeric materials, such as polyanhydrides and copolymers poly(lactic acid) grafted to amino acids; and international patent application WO 03/043586 discloses polymeric nanoparticles. Tartula et al (J. Drug Target., 19, 900 (2011)), Li et al (Nanomedicine, 11, 1377 (2015) and Wang et al, Nanoscale Research Letters, 12, 66 (2017)) disclose mesoporous silica nanoparticles for inhaled delivery of drugs. Finally, international patent application WO 03/011251 discloses mesoporous silicon carriers for pulmonary delivery.

Inhalation devices that are typically employed to administer active compounds to the lung include metered dose inhalers (MDIs), dry powder inhalers (DPIs) and soft mist inhalers (SMIs). DPIs may be divided into low, medium and high resistant DPIs.

The efficiency of DPIs, for example, is affected by two main forces 1) an inspiration air flow (IAF), which depends on a flow generated by the patient, and 2) a turbulence produced by the device.

A balance between these two forces is important for optimal performance of a device. If the IAF is too low, most of the drug is lost in the upper lung, i.e. the throat and the trachea. On the other hand, with most DPI-administered formulations, if the IAF is too high, more drug may be delivered in the lower lung (the bronchi and alveoli), but in a manner where there is often poor disaggregation of particles, and therefore dispersion of the drug in the lung.

Typical fixed-dose drug combinations for pulmonary delivery require powder homogeneity to deliver a uniform dose of drug to patients. This is often attempted by a simple blend of micronized drugs with coarse carrier particles.

In a MDI, the pharmaceutical composition is typically present in a liquid form, as a solution or suspension in a propellant, such as a hydrocarbon, a fluorocarbon or a hydrogen-containing fluorocarbon. In such systems it is often difficult to prevent dissolution of a bioactive compound from the particle or to prevent leakage of the compound from the drug-containing particle.

Typically, solvents and/or surfactants are employed with a view to imparting stability to the suspension of drug particles. The compound needs to have a low solubility in the solvents that are used.

In attempting to formulate specific mesoporous silica materials with specific properties with a view to inclusion of C21 for pulmonary delivery, an undesirable agglomeration resulted, which could not be alleviated with conventional lubricant excipients. Surprisingly, incorporation of C21, into the resultant silica particles resulted not only in deagglomeration of the resultant loaded particles, which was not expected, but also a chemically-stable solid state formulation, something that had never been obtained previously. This renders the resultant loaded silica particles of potential utility in the pulmonary delivery of such active ingredients.

We have also found that C21 shows an excellent solubility profile when presented in this way, which renders such compositions of potential utility in the topical treatment of ILDs, such as IPF, by pulmonary administration.

### Disclosure of the Invention

According to the invention, there is provided a pharmaceutical composition suitable for administration to the lung, which composition comprises a plurality of amorphous nanoporous (mesoporous) silica particles, in which C21 or a pharmaceutically-acceptable salt thereof is loaded into the pores of said silica particles, and wherein the silica particles have:
(a) a mass median aerodynamic diameter (MMAD) that is between about 0.1 μm and about 15 μm (e.g. about 10 μm); and
(b) a geometric standard deviation (GSD) that is less than about 4.

Such compositions are hereinafter referred hereinafter together as “the compositions of the invention”.

The loaded silica particles of the compositions of the invention may also have a mass density that is less than about 1 g/cm³, such as less than about 0.6 g/cm³, including less than about 0.4 g/cm³, for example between about 0.15 and about 0.35 g/cm³.

Mass median aerodynamic diameter (MMAD) will be understood by those skilled in the art to mean the diameter at which 50% of the particles by mass are larger and 50% are smaller over the total delivered dose as determined by any approved device, usually a cascade impactor such as a NGI, Andersen or Marple Miller impactor (see e.g. US Pharmacopeia at <601>; and/or www.uspbpep.com/usp31/v31261/usp31nf26s1_c601.asp). MMAD may be readily determined by those skilled in the art, for example by plotting on log probability paper the percentages of mass that is less than the stated aerodynamic diameters versus the aerodynamic diameters. The MMAD is taken as the intersection of the line with the 50% cumulative percent.

MMAD of the particles may be varied depending on the preferred and/or intended site of delivery of C21, or the pharmaceutically-acceptable salt thereof. A MMAD that may be mentioned is between about 5 μm and about 15 μm.

However, it is preferred that the MMAD of particles in compositions of the invention is between about 0.5 μm and about 8 μm, such as between about 1 μm and about 7 μm, for example between about 2 μm and about 6 μm, more preferably between about 3 μm and about 5 μm, such as between about 2.5 μm and about 4.8 μm, including between about 2.8 μm and about 4.4 μm, for example about 2.9 μm, about 3.0 μm, about 3.1 μm, about 3.2 μm, about 4.0 μm, about 4.1 μm, about 4.2 μm, or about 4.3 μm, more preferably between about 3.3 μm and about 3.9 μm, such as 3.4 μm or about 3.8 μm, including about 3.5 μm, about 3.6 μm or about 3.7 μm.

In the alternative, it is preferred that the MMAD of particles in compositions of the invention is between about 0.95 μm and about 6.05 μm; and, such as between about 1 μm and about 5.50 μm or, more preferably the MMAD of particles in compositions of the invention is between about 1.05 μm (e.g. about 1.5 μm) and about 5.0 μm, such as up to about 4.4 μm, for example up to about 3.0 μm, and for example specifically about 1.6 μm, about 1.7 μm, about 2.9 μm, about 2.8 μm, about 2.7 μm, about 2.6 μm, about 2.5 μm, about 2.4 μm, or about 2.3 μm preferably between about 1.8 μm and about 2.2 μm, such as 1.9 μm or about 2.1 or about 2.0 μm.

This will mean that particles will tend to deposit primarily in the bronchioli.

GSD will be understood by those skilled in the art to be a measure of the spread of an aerodynamic particle size distribution. It is typically calculated as follows as:

(d₉₀/d₁₀)^{1/2}

wherein d90 and d₁₀ represent the diameters at which 90% and 10%, respectively, of the aerosol mass are contained, in diameters less than these diameters.

It is preferred that the GSD of particles in compositions of the invention is less than about 2.5, such as less than about 2.2, e.g. less than about 2.0, including less than about 1.8, or more preferably less than about 1.5, such as between about 1 and about 1.5.

In the alternative, is preferred that the GSD of particles in compositions of the invention is no more than 1.33, such as about less than 1.32, such as less than 1.28 or less than 1.25, including less than 1.23. less than about 1.22, and even less than about 1.21, about 1.20, about 1.19, about 1.18, about 1.17, about 1.16, about 1.15, about 1.14, about 1.13, about 1.12, about 1.11, about 1.10, about 1.09, about 1.08, about 1.07, about 1.06 or about 1.05 or less.

Other parameters that may be used to define particles include mass density and the fine particle fraction (FPF). The FPF is the proportion of particles that have a diameter below about 5 μm. Preferred FPFs are at least about 50%, including at least about 60%, such as at least about 75% (e.g. at least about 80%), including at least about 85%, e.g. at least about 90%, such as at least about 95%, at least about 98%, and up to at least about 99%, at least about 99.9% or about 100%.

The silica particles that are employed in compositions of the invention may also have a pore size that is between about 1 nm (e.g. about 2 nm) and about 100 nm (e.g. about 50 nm). Porous silica particles of compositions of the invention preferably have an average pore size that is in the range of about 2 nm (e.g. about 3 nm, such as about 4 nm, including about 5 nm and about 8 nm) up to about 30 nm (e.g. about 20 nm, such as about 16 nm (e.g. about 15 nm), including about 13 nm, such as about 12 nm (e.g. about 10 nm). Specific average pore sizes that may be mentioned include about 4.5 nm, about 5.0 nm, about 5.5 nm, about 6.5 nm, about 7.0 nm, about 7.5 nm, about 8.0 nm, about 8.5 nm, about 9.0 nm, about 9.5 nm, about 10 nm, about 11 nm, about 12 nm, about 13 nm, or about 14 nm. Such particles may also possess a pore volume that is between about 0.05 cm³/g, such as about 0.08 cm³/g, including about 0.09 cm³/g (e.g. about 0.1 cm³/g, such as about 0.2 cm³/g, or about 2 cm³/g) and about 3 cm³/g, such as about 2.5 cm³/g, including about 2.0 cm³/g (e.g. about 1.5 cm³/g or about 1.0 cm³/g), and/or may preferably possess a surface area that is in the range of about 35 m²/g, e.g. about 40 m²/g or about 50 m²/g (such as about 100 m²/g, including about 150 m²/g or about 200 m²/g) up to about and about 1,200 m²/g, such as about 450 m²/g, including about 350 m²/g, e.g. about 300 m²/g. All of these parameters may be determined by routine techniques, such as nitrogen adsorption isotherm (Brunauer, Emmett and Teller (BET)), mercury inclusion, and Barrett, Joyner and Halenda (BJH), methods.

Shapes of the porous particles may be controlled by the process of manufacture. Shape may be important for the incorporation and dissolution of C21 or salt thereof. Thus, although silica particles may potentially be any shape (e.g. gyroids, rods, fibers, pseudo-spheres, cylinders, core-shells) in compositions of the invention, they are preferably essentially spherical. By “essentially spherical”, we mean that they may possess an aspect ratio smaller than about 20, more preferably less than about 10, such as less than about 4, and especially less than about 2, and/or may possess a variation in radii (measured from the centre of gravity to the particle surface), in at least about 90% of the particles that is no more than about 20% of the average value, such as no more than about 10% of that value, for example no more than about 5% of that value.

Porous silica particles may be loaded with C21, or a pharmaceutically-acceptable salt thereof, by any suitable process known to those skilled in the art. For example, particles may be loaded by way of a solvent evaporation technique, for example as described hereinafter, impregnation, for example using a melt, use of supercritical CO₂, shear mixing, co-grinding, spray-drying or freeze-drying. Well known equipment, such as a fluidized bed may be used. A preferred technique is solvent evaporation.

Loading C21 or pharmaceutically-acceptable salt thereof, into the silica particles means that the compound in question is loaded into the nanopores of the particles. It is preferred that the pores of the silica particles are loaded such that between about 0.1 and about 60% (e.g. about 50%), preferably up to about 45%, such as up to about 40%, such as up to about 35%, including up to about 30% or up to about 25% (e.g. about 20% or about 10%) of the total weight of the loaded particles is C21 or salt thereof, and, optionally, other pharmaceutical excipients, diluents or additives. In the alternative, it is preferred that up to about 60%, including up to about 70%, or up to about 80%, such as up to about 90%, e.g. up to about 100% of the pores of the silica particles are loaded with C21 or salt thereof and, optionally, other pharmaceutical excipients, diluents or additives. The entire mass of active compound does not have to be loaded into the pores of the particles and may otherwise be attached to the surfaces of the particle.

It is further preferred that C21, or salt thereof, is presented within the pores of the particles of compositions of the invention in an essentially amorphous state. By “essentially amorphous”, we mean that the active ingredient is no more than about 5%, such as no more than about 2%, for example no more than about 1%, e.g. no more than about 0.5%, and preferably no more than about 0.1% crystalline.

Presenting C21, or salt thereof, in an amorphous state within the pores of the particles of compositions of the invention means that the latter are capable of delivering a consistent and/or uniform dose of active ingredient, which is independent of solubility, after administration to the lung. We have found that, by incorporating C21, or salt thereof, into the pores of the particles of the compositions of the invention, it is possible to ensure that the active ingredient remains in the amorphous state during and after manufacture, under normal storage conditions, and during use.

By this, we include that C21, or pharmaceutically-acceptable salt thereof, can be stored in the form of a composition of the invention, optionally in admixture with pharmaceutically acceptable carriers, diluents or adjuvants, under normal storage conditions, with an insignificant degree of solid state transformation (e.g. crystallisation, recrystallisation, loss of crystallinity, solid state phase transition, hydration, dehydration, solvatisation or desolvatisation). In addition to this, C21 and pharmaceutically-acceptable salts thereof, may be stored in this form under normal storage conditions, with an insignificant degree of chemical degradation or decomposition.

Examples of “normal storage conditions” include temperatures of between minus 80 and plus 50° C. (preferably between 0 and 40° C. and more preferably ambient temperature, such as between 15 and 30° C.), pressures of between 0.1 and 2 bars (preferably atmospheric pressure), relative humidities of between 5 and 95% (preferably 10 to 60%), and/or exposure to 460 lux of UV/visible light, for prolonged periods (i.e. greater than or equal to six months). Under such conditions, C21, or a salt thereof, may be found to be less than about 15%, more preferably less than about 10%, and especially less than about 5%, solid-state and/or chemically transformed. The skilled person will appreciate that the above-mentioned upper and lower limits for temperature and pressure represent extremes of normal storage conditions, and that certain combinations of these extremes will not be experienced during normal storage (e.g. a temperature of 50° C. and a pressure of 0.1 bar).

In addition to the fact that it is often preferred to present active ingredients in an amorphous form to enhance dissolution and/or systemic absorption, one of the biggest challenges facing pharmaceutical formulators in the field of drug delivery to the lung is that the adhesion of microparticulate active ingredients to a carrier in, say, a DPI device is highly influenced by crystallinity, which can change over time.

In particular, active ingredient can be transformed from one solid state form to another, resulting in changes in adhesive forces, which will, in turn, affect the performance of the formulation to be inhaled.

To solve this problem, formulators have often presented microparticulate active ingredients in a crystalline form in such prior art formulations. This has often led to difficulties in achieving reproducibly, because micronization or milling are often used to reduce particle size of the active ingredient, which can lead to high energy particles in amorphous form.

Thus, the loading of C21 or salts thereof into the pores of the silica particles in accordance with the invention physically stabilizes the active ingredient in an amorphous form and prevents it from crystalizing, meaning that the physio-chemical properties of the drug do not change over time

We prefer that the amorphous porous silica particles are biodegradable mesoporous silica.

The term “biodegradable” means that the silica particles are dissolvable. Accordingly, a preferred embodiment of the invention is that the silica is a synthetic amorphous silica.

In order to be completely dissolvable, the silica particles of the compositions of the invention must be amorphous and therefore entirely non-crystalline (and remain so under normal storage conditions as hereinbefore defined), by which we mean that no crystallinity is detectable by standard techniques, such as XPRD. This is especially important considering the indications in which the compositions of the invention are intended to be used, in which injury by crystalline silica or other agents may be one of the causes of ILDs, such as PF.

Amorphous silica is less sensitive to humidity when compared to dry crystalline powder compositions that are typically used in pulmonary delivery of active ingredients.

Amorphous silica particles may be manufactured by any process known in the art.

In one embodiment, porous particles may be manufactured by cooperative self-assembly of silica species and organic templates such as cationic surfactants such as alkyltrimethylammonium templates with varying carbon chain lengths, and counterions such as cetyltrimethylammonium chloride (CTA+Cl— or CTAC) or cetyltrimethylammonium bromide (CTA+Br— or CTAB), or non-ionic species such as diblock and triblock polymer species, such as copolymers of polyethylene oxide and polypropylene oxide for example Pluronic 123 surfactant.

The formation of mesoporous silica particles occurs following the hydrolysis and condensation of silica precursors which can include alkylsilicates such as tetraethylorthosilcate (TEOS) or tetramethylorthosilicate (TMOS) in solution or sodium silicate solution. The mesoporous silica particle size can be controlled by adding suitable additive agents, e.g. inorganic bases, alcohols including methanol, ethanol, propanol, and other organic solvents, such as acetone, which affect the hydrolysis and condensation of silica species.

Pore size may not only be influenced by hydrothermal treatment of the reaction mixture such as heating up to 100° C. or even above and also with the addition of swelling agents in the form of organic oils and liquids that expand the surfactant micelle template, but also, after condensation of the silica matrix, removing the templating surfactant by calcination typically at temperatures from about 500° C. to about 650° C., or alternatively from 650° C. up to about 750° C., in each case for e.g. several hours. Calcination at the higher of the above two temperature ranges not only burns away the organic template resulting in a porous matrix of silica (which the lower of the above two temperature ranges will also achieve), but also creates particles with one or more of the smaller average pore sizes mentioned hereinbefore (e.g. about 2 nm to about 14 nm, about 3 nm to about 13 nm and/or about 4 nm to about 12 nm (e.g. about 10 nm)), pore volumes (e.g. about 0.05 cm³/g to about 2.5 cm³/g, including about 0.08 cm³/g (e.g. about 0.09 cm³/g) up to about 2.5 cm³/g, including about 2 cm³/g (e.g. about 1.5 cm³/g, such as 1.0 cm³/g)), and/or surface areas mentioned herein (e.g. about 35 m²/g to about 450 m²/g, and/or about 50 m²/g (e.g. about 100 m²/g, including about 200 m²/g) and about 400 m²/g, such as about 350 m²/g)). The template may alternatively be removed by extraction and washing with suitable solvents such as organic solvents or acidic of basic solutions.

In another embodiment, the porous silica particles may be manufactured by a sol-gel method comprising a condensation reaction of a silica precursor solution, such as sodium silicate or an aqueous suspension of silica nanoparticles as an emulsion, with a non-miscible organic solution, oil, or liquid polymer in which droplets are formed by for example stirring or spraying the solution, followed by gelation of the silica by means of change in pH and or evaporation of the aqueous phase.

The porosity of the particles here are formed either by exclusion due to the presence of the non-miscible secondary phase or by the jamming of the silica nanoparticles during evaporation.

Such particles may further be treated by heating to induce condensation of the silica matrix and washing to remove the non-miscible secondary phase. Furthermore, the particles may be treated by calcination as hereinbefore described to strengthen the silica matrix.

In another embodiment, the porous particles may be manufactured as porous glass through a process of phase separation in borosilicate glasses (such as SiO₂—B₂O₃—Na₂O), followed by liquid extraction of one of the formed phases through the sol-gel process, or simply by sintering glass powder. During a thermal treatment, typically between 500° C. and 760° C., an interpenetration structure is generated, which results from a spinodal decomposition of the sodium-rich borate phase and the silica phase.

The porous particles may also be manufactured using a fumed process. In this method, fumed silica is produced by burning silicon tetrachloride in an oxygen-hydrogen flame producing microscopic droplets of molten silica which fuse into amorphous silica particles in three-dimensional secondary particles which then agglomerate into tertiary particles. The resulting powder has an extremely low bulk density and high surface area.

In view of the particle size of the silica particles of the compositions of the invention, particle aggregation was expected. Aggregation of dry particles in the micron-sized range is a well-known phenomenon in particle and powder processing. Aggregation is caused by numerous attractive (ubiquitous) forces, such as van der Waals forces and/or electrostatic interactions. In many cases, particle aggregation causes unwanted problems such as poor handling and flowability and sticking to containers.

Particles within the size range mentioned herein are also often prone to aggregation in air due to the large surface area to volume ratio.

In view of the above, particle aggregation is a serious hurdle for pulmonary delivery, given that the particle size is critical to ensure correct distribution of the particles in the lung. Aggregation of particles would be expected to lead to accumulation in the throat and upper airways thereby limiting the effectiveness of the formulation. Additionally, aggregation of particles or sticking of particles in the capsules during inhalation is a severe limitation.

Indeed, in the field of inhalation of dry powders, for example active ingredients are typically administered in the form of micronized particles (of a size between about 1 and about 6 μm). The problem of aggregation is typically solved by either suspending micronized particles of active ingredient in a propellant (e.g. HFA), sometimes with other excipients, such as mannitol, lactose, sorbitol, etc.) in an MDI requiring actuation, or by blending such micronized particles of active ingredient with an inactive excipient of larger particle size (e.g. mannitol or lactose), inside a capsule, which is then pre-loaded or manually loaded into a DPI, whereupon inhalation de-aggregates the medication particles and disperses them within the airways.

Furthermore, although monodisperse porous particles in the size range 1 to 6 μm may be made on a bench scale by various routine methods, such as precipitation, seeded and controlled growth, emulsification and microfluidics techniques, it is very difficult or very costly to manufacture porous particles on a larger, industrially-relevant scale with a perfectly uniform particle size without the creation of fine particles or sub-micron particles. Spray drying is technique that is used on an industrial scale but this produces particles with a broad particle size distribution and creates fine particles.

As described hereinafter, when small amounts of pre-loaded silica particles that might be considered to be suitable for inhalation were investigated, as expected, the particles aggregated and had poor flow properties. When attempts were made to use common excipients that are normally employed to reduce particle aggregation, such as lubricants, the flow properties of the particles did not improve.

However, as also described hereinafter, when the same particles were loaded with C21, the flow properties of the particles were improved without the addition of further excipients.

The silica particles may be manufactured by one or more of the processes described hereinbefore to a specification that has the MMAD and/or GSD (as well as other parameters) within any of the ranges or limits described herein.

Alternatively (or preferably), following such manufacture and prior to loading with C21 or salt thereof, they may be separated and classified into the particle size ranges disclosed herein by an appropriate process known to those skilled in the art. In this respect, particles may be separated using cyclonic separation, by way of an air classifier, sedimentation, force-field fractionation, and/or by sieving using one or more sieves or filters to obtain particles within the desired size ranges. However, we have found that particles within a desired size ranges are preferably obtained by elutriation, for example as described hereinafter.

Elutriation is a process for separating particles based on their size, shape and density, using flowing liquid in a direction opposite to the direction of sedimentation. The smaller particles rise to the top (overflow) because their terminal sedimentation velocities are lower than the velocity of the rising fluid.

We have also found that the use of elutriation to separate mesoporous silica particles results in the production of such particles with:
(i) MMADs that are between about 0.95 μm and about 6.05 μm; such as between about 1 μm and about 5.50 μm or, more preferably between about 1.05 μm (e.g. about 1.5 μm) and about 5.0 μm, such as up to about 4.4 μm, for example up to about 3.0 μm, and for example specifically about 1.6 μm, about 1.7 μm, about 2.9 μm, about 2.8 μm, about 2.7 μm, about 2.6 μm, about 2.5 μm, about 2.4 μm, or about 2.3 μm preferably between about 1.8 μm and about 2.2 μm, such as 1.9 μm or about 2.1 or about 2.0 μm; and/or (ii) GSDs that are no more than 1.33, such as about less than 1.32, such as less than 1.28 or less than 1.25, including less than 1.23. less than about 1.22, and even less than about 1.21, about 1.20, about 1.19, about 1.18, about 1.17, about 1.16, about 1.15, about 1.14, about 1.13, about 1.12, about 1.11, about 1.10, about 1.09, about 1.08, about 1.07, about 1.06 or about 1.05 or less,
   thus, particles that are very small, but have a well-defined particle size distribution.

We have surprisingly found that producing particles with a well-defined particle size distribution by the substantial removal of fines prior to loading with C21 or salt thereof, and so providing particles with the MMADs and GSDs as described above, results in a particles that do not aggregate in the expected (and previously observed) manner. This renders the resultant silica particles of potential utility for pulmonary delivery prior to and after being loaded with C21 or salt thereof.

According to a further aspect of the invention, there is provided a pharmaceutical composition suitable for administration to the lung, which composition consists essentially of a plurality of amorphous nanoporous (mesoporous) silica particles, in which C21 or a pharmaceutically-acceptable salt thereof is loaded into the pores of said particles, and wherein the silica particles have:
(a) a mass median aerodynamic diameter (MMAD) that is between about 0.95 μm and about 6.05 μm; and
(b) a geometric standard deviation (GSD) that is no more than 1.33.

In this context, the phase ‘consisting essentially of’ includes that the composition of the invention is substantially free of any excipients that are added and either do act, or are intended to act, as a lubricant. Thus, the phrase ‘consisting essentially of’ includes that the composition comprises less that about 1%, such as less than about 0.5%, including less than about 0.1%, or less than about 0.01%, or even less than about 0.001%, of such excipients by total weight of a composition of the invention.

Previously in the field of powders comprising small particles (e.g. microparticles), those skilled in the art have typically added smaller particles in order to improve flowability. In addition to lubricants that are often employed, smaller particles of e.g. colloidal silica or silica nanoparticles have been added to particulate compositions with a view to reducing the contact area between primary microparticles and so to improve the flowability of those primary particles as a powder (see, for example, Yang et al, Powder Technology, 158, 21 (2005)). It is therefore a real surprise that we have been able to prevent aggregation by removing smaller silica particles in the form of fines.

The process described herein allows for the removal of fines, and thereafter the production of compositions of the invention on an industrial scale, which compositions are provided in the form of handleable powders that can be administered to patients using conventional devices in a manner that provides effective therapy in a reproducible manner, in view of the fact that aggregation is not a problem.

In addition, prior to prior to loading with C21 or salt thereof, silica particles may be surface modified by chemical reaction of free silanol groups with a reagent that provides at least one organic group.

This can be achieved by surface modification of silica particles by reaction with an alkoxysilane, and/or an alkylhalosilane, many of which are commercially available, for example as described hereinafter. These reagents are capable of forming 1 to 3 Si—O—Si links to the surface by way of a condensation reaction with surface silanol groups.

Typical functionalising reagents that may be employed to achieve this include 3-aminopropyl triethoxysilane, 3-mercaptopropyl trimethoxysilane and various PEG-silanes, but we prefer that the functionalising reagent is an alkylhalosilane, which alkylhalosilane may be an alkylchlorosilane containing up to 4 (e.g. 3) alkyl groups, such as between 1 and 4 (e.g. 3) 0₁-₂₄ alkyl groups, such as C₁₋₁₈ alkyl groups, including C₁₋₁₀ alkyl groups, e.g. a di- or tri-C₁₋₄ alkylchlorosilane, such as tripropylchlorosilane, triethylchlorosilane or, most preferably, trimethylchlorosilane.

Surface modifications of this type may be carried out by reacting said functionalising reagent with silica particles, optionally in the presence of an appropriate solvent (e.g. toluene) and/or an appropriate base (e.g. imidazole), for example as described hereinafter, alternatively as described in Zhao and Li, J. Phys. Chem., 102, 1556 (1998) or Taib et al, Int. J. Chem., 3, 2 (2011).

Further, after loading with C21 or salt thereof, the loaded silica particles may be admixed with one or more fatty acid- or lipid-based surfactants. Such admixing is preferably done by dry mixing said surfactant with said loaded particles, more preferably by way of a high energy mixing process. Appropriate high energy mixing equipment may include, for example, intensive mechanical processors (e.g. the Nobilita-130 Unit Mechanofusion System (Hosokawa Micron Corporation, Osaka, Japan)), for example under appropriate mixing conditions, such as those described in Zhou et al, J. Pharm. Sci., 99, 969 (2010).

Admixing may also be achieved by other techniques known to those skilled in the art, including spraying a solution or a suspension of said surfactant onto the surfaces of said particles by a suitable means.

The term ‘fatty acid- or lipid-based surfactant’ will be understood to include any surfactant comprising a long (C₈-₂₄) hydrocarbon chain. Surfactants comprising such hydrocarbon chains are or may be derived from oilseeds (e.g. palm, palm kernel, coconut, etc.), and may be saturated, branched, linear and/or aromatic. Surfactants based on lipids or fatty acids may be non-ionic, but are preferably ionic.

Ionic surfactants may include those with a cationic head group (e.g. primary, secondary, or tertiary amines; primary and secondary amines and quaternary ammonium salts); Zwitterionic (amphoteric) surfactants (e.g. sultaines, betaines and phospholipids); but more preferably include anionic surfactants, such as salts of sulfate esters (e.g. ammonium lauryl sulfate and sodium lauryl sulfate), sulfonate esters and phosphate esters or, more preferably, carboxylate esters.

Anionic surfactants based on carboxylate esters include carboxylate salts (soaps), which surfactants comprises an alkali, or an alkaline earth, metal ion (e.g. sodium, potassium, calcium or magnesium) and one or more fatty acid chain with at least 10, such as at least 12, including at least 14, such as at least 16, carbon atoms. Preferred specific anionic surfactants in this class include sodium stearate, sodium lauroyl sarcosinate and carboxylate-based fluorosurfactants, such as perfluorononanoate and perfluorooctanoate. However, we prefer that the surfactant that is employed in compositions of the invention is magnesium stearate.

The amount of surfactant that may be employed in compositions of the invention is in the range of about 0.1% to about 12% by weight of the composition, such as about 0.2% to about 11%. Preferred amounts are in the range of about 1%, such as about 2%, including about 3%, up to about 10% by weight of the composition. Specific amounts that may be included are thus about 0.5%, about 1.0%, about 1.5%, about 2.0%, about 2.5%, about 3.0%, about 3.5%, about 4.0%, about 4.5%, about 5.0%, about 5.5%, about 6.0%, about 6.5%, about 7.0%, about 7.5%, about 8.0%, about 8.5%, about 9.0%, about 9.5%, about 10.0% and about 10.5%.

According to a further aspect of the invention there is a provided a process for the production of a composition of the invention, which process comprises:
(a) forming mesoporous silica particles by a method as described herein;
(b) optionally, calcining said silica particles at a temperature of between about 500° C. and about 600° C., or about 650° C. and about 750° C., in order to provide particles having relevant pore sizes, pore volumes, and/or surface areas, within the ranges described herein;
(c) if particles formed by steps (a) and/or (b) do not, at least in part, have an MMAD and/or a GSD within the ranges specified herein, separating said particles as described herein, so as to obtain particles having an MMAD and/or a GSD within those ranges;
(d) optionally surface modifying said silica particles by chemical reaction of the particles and in particular the free surface silanol groups with a reagent that provides at least one organic group, as described hereinbefore;
(a) loading the obtained silica particles with C21 or a pharmaceutically-acceptable salt thereof, as described herein; and
(b) optionally, admixing the loaded particles from step (e) with a fatty acid- or lipid-based- surfactant as described herein.

The process described herein for production of compositions of the invention has the advantage that it allows the production of particles with sizes that enable better control of the site of deposition of the particles in the lung, so enabling accurate tailoring of site-specific lung delivery (e.g. improved delivery to the deep lung) compared to prior art inhalation formulations comprising other drugs. The process described herein also reduces manufacturing costs compared to processes in which separation is conducted after loading particles with a bioactive compound. This may improve the yield and efficiency of the manufacturing process. The process also provides for a higher drug loading of the bioactive compounds in final compositions of the invention.

Thus, compositions of the invention have unexpectedly good flow properties which renders them suitable for pulmonary delivery. In this respect, compositions of the invention find particular utility in the pulmonary treatment of an ILD. In this respect, there is provided a composition of the invention for use in the treatment of an ILD by pulmonary administration as well as the use of a composition of the invention for the manufacture of a medicament for the treatment of an ILD by pulmonary administration.

The term “ILD” will be understood by those skilled in the art to include any pulmonary condition characterized by an abnormal healing response, including chronic inflammation, reduced lung function and/or scarring, irrespective of the cause, such as sarcoidosis, and PF, especially IPF. The term may also include diseases and/or conditions that are known to lead to, and/or be causes of, such pulmonary conditions, such as systemic sclerosis. In this respect there is further provided a composition of the invention for use in the condition that leads to and/or is a cause of an ILD, such as PF or IPF, including systemic sclerosis.

According to a further aspect of the invention there is provided a method of treatment of an ILD, which method comprises the pulmonary administration of a pharmacologically-effective amount of C21, or a pharmaceutically-acceptable salt thereof, in the form of a composition of the invention to a patient in need of such treatment.

“Patients” include mammalian (particularly human) patients. Human patients include both adult patients as well as paedeatric patients, the latter including patients up to about 24 months of age, patients between about 2 to about 12 years of age, and patients between about 12 to about 16 years of age. Patients older than about 16 years of age may be considered adults for purposes of the present invention. These different patient populations may be given different doses of C21, or salt thereof.

Pharmaceutically-acceptable salts of C21 include base addition salts and preferably acid addition salts. Such salts may be formed by conventional means, for example by reaction of a free acid or, preferably, free base form of an active ingredient with one or more equivalents of an appropriate acid or base as appropriate, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of an active ingredient in the form of a salt with another counter-ion, for example using a suitable ion exchange resin. Preferred salts of C21 include acid addition salts, such as HCl salts, alkaline earth salts, such as magnesium and calcium salts, and alkali metal salts, such as potassium or, preferably, sodium salts.

Although C21 and salts thereof may possess pharmacological activity as such, certain pharmaceutically-acceptable (e.g. “protected”) derivatives of C21 may exist or be prepared which may not possess such activity, but may be administered and thereafter be metabolised in the body to form C21. Such compounds (which may possess some pharmacological activity, provided that such activity is appreciably lower than that of C21 may therefore be described as “prodrugs” of C21.

As used herein, references to prodrugs will include compounds that form a compound of the invention, in an experimentally-detectable amount, within a predetermined time, following administration. All prodrugs of C21 are included within the scope of the invention.

Pulmonary delivery means compositions of the invention are adapted for delivery to the lungs by direct inhalation, and thereby giving rise to the direct topical treatment by C21 or salts thereof of ILDs in the lungs.

Administration of C21 or salt thereof is preferably intermittent. The mode of administration may also be determined by the timing and frequency of administration, but is also dependent, in the case of the treatment of ILDs, on the severity of the condition.

Compositions of the invention may also impart, or may be modified to impart, an immediate, or a modified, release of active ingredient(s).

Compositions of the invention may be combined with other excipients that are well known to those skilled in the art for pulmonary delivery of active ingredients. For example, optional excipients may include propellants; surfactants, such as poloxamers; if needed, glidants/lubricants, such as magnesium stearate or silica nanoparticles; sugars or sugar alcohols, such as lactose, glucose, mannitol or trehalose; lipids, such as DPPC, DSPC, DMPC, cholesterol; amino acids, such as leucine or trileucine; cyclodextrins, hydroxypropylated chitosan, poly-lactic-co-glycolic acid (PLGA); antioxidants; humidity regulators and the like, though such are by no means essential. Indeed, we have found that, in the pulmonary delivery of compositions of the invention, fewer additional excipients are needed, which may reduce cost of manufacture.

Inhalation devices that may be employed to administer compositions of the invention to the lung include MDIs, SMIs and DPIs, including low, medium and high resistant DPIs.

Compositions of the invention may form stable compound suspensions when suspended in solvents that are typically employed in MDIs. The loaded silica particles may be well-dispersed in different solvents and may be further modified to prevent dissolution or leakage of drug into the solvent before delivery to the target site or lung.

In view of the fact that, as mentioned hereinbefore, compositions of the invention have unexpectedly good flow properties, this minimizes the need for disaggregation of the particles by increased IAF and turbulence produced by the inhalation device. This in turn improves that the balance between the two forces discussed hereinbefore, and thus improves delivery of active ingredient to the lower lung without loss of drug in the upper lung. This further reduces the dependence on the inhalation device that is employed.

According to a further aspect of the invention, there is further provided a drug delivery device adapted for delivery of active ingredients to the lung, which delivery device comprises a composition of the invention.

The delivery device may be a MDI, a DPI or a SMI. When used in, in particular, a MDI, the composition of the invention is optionally mixed with a propellant, which propellant has a sufficient vapour pressure to form aerosols upon activation of the delivery device. The propellant may be selected from the group a hydrocarbon, a fluorocarbon, a hydrogen-containing fluorocarbon and a mixture thereof.

The above-mentioned excipients may be commercially-available or otherwise are described in the literature, for example, Remington The Science and Practice of Pharmacy, 19th ed., Mack Printing Company, Easton, Pa. (1995) and the documents referred to therein, the relevant disclosures in all of which documents are hereby incorporated by reference. Otherwise, the preparation of suitable pulmonary formulations may be achieved non-inventively by the skilled person using routine techniques.

Similarly, the amount of C21 or salt thereof in the formulation will depend on the severity of the condition, and on the patient, to be treated, but may be determined by the skilled person.

For example, suitable lower daily doses (calculated as the free base) of C21 in adult patients (average weight e.g. 70 kg), may be about 0.01 mg, such as about 0.1 mg, for example about 1 mg, or about 5 mg, per day. Suitable upper limits of daily dose ranges of C21 may be about 600 mg, including about 400 mg and about 200 mg, such as about 50 mg, including about 25 mg, such as about 10 mg.

All of the above doses are calculated as the free base and, again, doses may be split into multiple individual doses per day. Inhaled doses may be given between once and six, such as four times daily, preferably three times daily and more preferably twice daily. Alternatively, inhaled doses may be given between once and four times weekly, for example every other day.

In any event, the medical practitioner, or other skilled person, will be able to determine routinely the actual dosage, which will be most suitable for an individual patient, depending on the severity of the condition and route of administration. The above-mentioned dosages are exemplary of the average case; there can, of course, be individual instances where higher or lower dosage ranges are merited, and such are within the scope of this invention.

The dose administered to a mammal, particularly a human, in the context of the present invention should be sufficient to effect an appropriate response in the mammal (e.g. human) over a reasonable timeframe (as described hereinbefore). One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the age, condition, body weight, sex and response of the patient to be treated, and the stage/severity of the disease, as well as genetic differences between patients.

Although intended for inhalation, compositions of the invention may be co-administered with other pharmaceutical formulations comprising different (or the same) active ingredients that are intended for the treatment of ILDs (whether administered pulmonarily, orally, or otherwise). For example, uses and methods that involve pulmonary administration of compositions of the invention may be combined with one or more treatments comprising other active ingredients that are useful in the treatment of ILDs (or a peroral treatment comprising C21 or salt thereof).

Relevant active ingredients that may be used in combination therapy with C21 in the treatment of IPF include, for example, anti-fibrotics, such as nintedanib and pirfenidone; corticosteroids, such as cortisone and prednisone; inflammation suppressants, such as cyclophosphamide; other immunosuppressants, such as azathioprine and mycophenolate mofetil; and antioxidants, such as N-acetylcysteine. Relevant active ingredients that may be used in combination therapy with C21 in the treatment of sarcoidosis include, for example, corticosteroids, such as cortisone, prednisone and prednisolone; antimetabolites; immune system suppressants, such as methotrexate, azathioprine, leflunomide, mycophenoic acid/mycophenolate mofetil, cyclophosphamide; aminoquinolines; monoclonal anti-tumor necrosis factor antibodies, such as infliximab and adalimumab; immunomodulatory imide drugs, such as include lenalidomide, pomalidomide and, especially, thalidomide; the TNF inhibitor, etanercept; and painkillers, such as ibuprofen and paracetamol; cough suppressants and/or expectorants.

Relevant patients may thus also (and/or may be already) be receiving such therapy for the treatment of their ILD based upon administration of one or more of such active ingredients, by which we mean receiving a prescribed dose of one or more of those active ingredients mentioned herein, prior to, in addition to, and/or following, treatment with C21 or a salt thereof.

Pharmaceutically-acceptable salts, and doses, of other active ingredients useful in the treatment of ILDs include those that are known in the art and described for the drugs in question to in the medical literature, such as Martindale—The Complete Drug Reference (35th Edition) and the documents referred to therein, the relevant disclosures in all of which documents are hereby incorporated by reference.

Wherever the word “about” is employed herein, for example in the context of amounts, i.e. absolute amounts such as sizes (aerodynamic diameters, particle sizes and pore sizes), doses, weights or concentrations of (e.g. active) ingredients, pore volumes, particle surface areas, particle densities, temperatures or time periods; or relative amounts including percentages, standard deviations and aspect ratios, it will be appreciated that such variables are approximate and as such may vary by ±10%, for example ±5% and preferably ±2% (e.g. ±1%) from the actual numbers specified. In this respect, the term “about 10%” means e.g. ±10% about the number 10, i.e. between 9% and 11%.

In addition to the advantages mentioned hereinbefore, compositions of the invention provide for an improved drug loading for the reasons described hereinbefore. This enables high quantities/doses of bioactive compound to be presented in dosage forms comprising compositions of the invention, and also efficient delivery of such higher doses to the desired site in the lung in a consistent/uniform manner. This in turn means that the frequency of dosing may be reduced and thus the effectiveness and efficiency of treatment as well as costs for healthcare reduced.

Furthermore, improved efficiency of deposition of C21 or salt thereof in the lung in view of the low amount of aggregation of particulates within which the active ingredient is loaded allows for more precise lung delivery, and thus an improved therapeutic effect.

Homogeneity in terms of both carrier particle size and drug distribution within the composition may also be improved by compositions of the invention.

In addition, if desired, compositions of the invention may include additional bioactive compounds (C21 or salt thereof, or otherwise as described hereinbefore), which may also be loaded into silica particles without substantial loss of material. This may be useful in e.g. co-therapy as described hereinbefore, and moreover may further reduce cost of manufacture.

Compositions of the invention also have the advantage that the dissolution kinetics of C21 or salt thereof, is largely independent of particle size, morphology of the compound and site of delivery in the lung. Adjusting pore size may thus be employed to tailor drug dissolution kinetics, but will be independent of the position of the particles in the lung.

The uses/methods described herein may otherwise have the advantage that, in the treatment of ILDs, they may be more convenient for the physician and/or patient than, be more efficacious than, be less toxic than, have a broader range of activity than, be more potent than, produce fewer side effects than, or that it may have other useful pharmacological properties over, similar methods (treatments) known in the prior art.

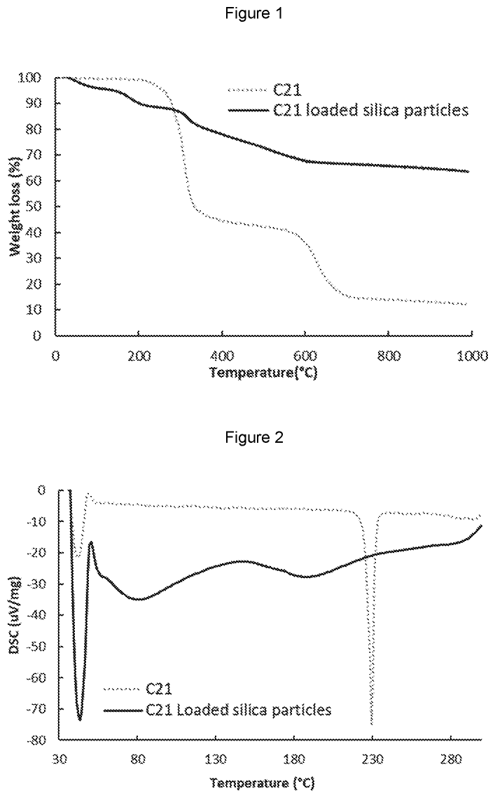

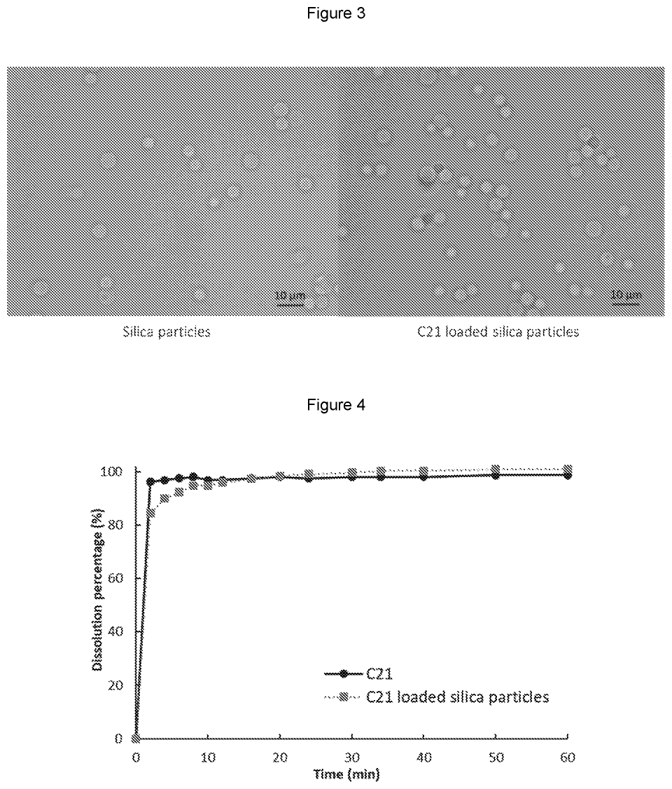

The invention is illustrated, but in no way limited, by the following examples, in which FIG. 1 is a thermogravimetric analysis (TGA) of silica particles loaded with C21, in the form of the sodium salt; FIG. 2 is a differential scanning calorimetry (DSC) analysis characterizing the physical state of C21 after being loaded into silica particles; FIG. 3 is a light microscope image showing free C21 (on the left) and C21 loaded into silica particles (on the right); and FIG. 4 shows inter alia an in vitro release profile for C21 released from silica particles in simulated lung fluid (SLF).

### EXAMPLES

### Example 1

### Silica Particle Manufacture I

Pluronic 123 (triblock co-polymer, E020P070E020, Sigma-Aldrich; 4 g; templating agent) and 1,3,5-trimethylbenzene (TMB; mesitylene, Sigma-Aldrich; 3.3 g; swelling agent) were dissolved in 127 mL of distilled H₂O₂and 20 mL of hydrochloric acid (HCl, 37%, Sigma-Aldrich) while stirring at room temperature for 3 days.

The solution was preheated to 40° C. before adding 9.14 mL of tetraethyl orthosilicate (TEOS; Sigma-Aldrich). The mixture was stirred for another 10 minutes at a speed of 500 rpm, then kept at 40° C. for 24 hours, and then hydrothermally treated in the oven at 100° C. for another 24 hours. Finally, the mixture was filtered, washed and dried at room temperature.

The product was calcined to remove the surfactant template and swelling agent. The calcination was conducted by heating to 600° C. with a heating rate of 1.5° C./min and kept at 600° C. for 6 hours, followed by cooling to ambient conditions. The resultant product was a white powder comprising porous silica particles.

### Example 2

### Silica Particle Manufacture II

A dispersion (14 wt %) of silica nanoparticles (10 nm) in water (pH 9) (400 mL) was poured into benzyl alcohol (800 mL) warmed to 50° C. and stirred at 300 rpm with an overhead stirrer (Silverson, UK) for 20 minutes.

A drop of acetic acid was added and vacuum (200 bar) was applied during heating at 80° C. to remove the aqueous phase. The resulting particles were collected by filtration and washing with acetone.

The product was calcined by heating to 600° C. with a heating rate of 1.5° C./min and kept at 600° C. for 6 hours, followed by cooling to ambient conditions. The resultant product was a white powder comprising porous silica particles in the size range 2 to 4 microns measured by scanning electron microscope (JEOL, Japan) and by electrical sensing zone method (Elzone, Micromeretics USA). The particles were further treated by refluxing in ammonium hydroxide overnight followed by filtering and refluxing in nitric acid overnight and finally filtered and washed in water and oven dried at 80° C.

### Example 3

### Silica Particle Separation

100 g of nanoporous silica particles (preparable and/or prepared as described in Example 1 and/or Example 2 above) were fed into an air classifier (TTS, Hosokawa-Alpine), with the air flow adjusted from 53 to 42 m³/h and the speed set at between 2,475 and 13,500 rpm. 11 g of fines and 8 g of course materials were collected. The particle size distribution calculated as (D₉₀/D₁₀) was reduced from 4.5 to 1.8.

### Example 4

### Nanoporous Silica Particle Properties

Two porous silica particle types with different porosities and densities (1.2 mL/g and 0.9 mL/g, and 0.18 mg/mL and 0.33 mg/mL, respectively) were prepared essentially as described in Example 1 and/or Example 2, and were characterised to determine their MMAD, GSD and GPS (mean particle size) using an 8-Stage Cascade Impactor (Marple), as shown in Table 1 below.

TABLE 1

| | Bulk | | | | | | |
|---|---|---|---|---|---|---|---|
| | Density | D₁₀ | D₅₀ | D₉₀ | MMAD | GPS | |
| | (mg/mL) | (μm) | (μm) | (μm) | (μm) | (μm) | GSD |
| Silica 1 | 0.18 | 3.0 | 4.1 | 5.5 | 4.33 | 3.8 | 1.354 |
| Silica 2 | 0.33 | 2.6 | 3.8 | 5.1 | 3.6 | 4.1 | 1.401 |

By optical observation, it could be seen that, in small quantities, the above particles aggregated and had poor flow properties.

Attempts were made to reduce particle aggregation for formulation by using the well-known glidant magnesium stearate. This is an approved excipient for inhalation products. The particles were mechanically mixed with magnesium stearate (Sigma) at several different weight ratios in the range 1-5% magnesium stearate, but the flow properties of the particles did not improve.

### Example 5

### Loading of C21 in Silica

C21 was encapsulated into the porous silica particles of Example 4 above (those with the bulk density of 0.18 mg/mL and the MMAD of 4.33 μm) by a solvent impregnation and evaporation method. A concentrated solution of C21 was made in a chosen good solvent for the drug, and various known masses of nanoporous silica particles were added to the solution. The solvent was removed by evaporation.

In one example, C21 as the sodium salt (450 mg) was dissolved in methanol (15 mL; VWR) at room temperature in a round-bottomed flask. Nanoporous silica particles (1050 mg) were added to the C21 solution.

The mixture was stirred for 30 minutes at 40° C. The solvent was evaporated with controlled evaporation under a reduced pressure of 200 mbar in a rotary evaporator, with a water bath temperature of 40° C. The resultant dry powder that was collected was free flowing. The samples were further dried at 40° C. under vacuum for 12 hours. The resulting loaded silica particles were free flowing and showed good handling properties.

The samples were characterized by thermogravimetric analysis (TGA) to evaluate drug loading. In FIG. 1, an analysis of mass loss from free C21 and C21-loaded silica particles is shown. The main degradation of C21 appears between 300 and 600° C. A loading amount of 30 wt % (calculated as mass of drug/mass of loaded particles) was determined.

The physical state of the drug (crystalline vs amorphous) was measured with differential scanning calorimetry (DSC) and is shown in FIG. 2. FIG. 2 shows DSC measurements of free C21 and C21 loaded into porous silica particles. C21 has a clear melting peak in DSC of around 230° C. It can be seen from FIG. 2 that the melting peak was completely absent when C21 was loaded into porous silica particles, showing that the C21 is stabilized in an amorphous state in the samples with a drug loading of up to 30 wt %.

Analysis by light microscopy showed that the free drug is fully encapsulated in the porous silica particles. This is shown in FIG. 3 (empty silica particles on the left, and C21 silica particles (30% loading) on the right). No C21 was detected around the outside of the silica particles.

### Example 6

### Dissolution Kinetics of C21-Loaded Silica Particles

The dissolution kinetics of the C21-loaded silica described in Example 5 were characterized in SLF (pH 7.4; Gamble's solution: made up with the salts NaCl, NaHCO₃, KCl, MgCl₂, CaCl₂, Na₂SO₄, sodium citrate dihydrate, sodium acetate, NaH₂PO₄ (all from Sigma-Aldrich)) at 37° C. (Reference: Simulated Biological Fluids with Possible Application in Dissolution Testing, Marques et al,. Dissolution technologies August 2011, p15-28) using a USP 2 dissolution apparatus with stirring speed 75 rpm. Free, unloaded C21-sodium salt was used as control. Concentration of drug at set times after release was measured by a UV/vis spectrometer (Cecil 3021) at 267 nm.

The dose of C21 was calculated as 25 mg of C21 in 500 mL of SLF. The data are shown in FIG. 4. The C21 sodium salt is readily soluble in SLF conditions and show immediate dissolution. The dissolution kinetics of free C21 shows a rapid dissolution profile with 96% released in the first 2 minutes. The loaded C21 is fully released and dissolved from the silica particles in around 10 minutes.

1. A pharmaceutical composition suitable for administration to the lung, which composition comprises a plurality of amorphous nanoporous (mesoporous) silica particles, in which N-butyloxycarbonyl-3-(4-imidazol-1-ylmethylphenyl)-5-iso-butylthiophene-2-sulfonamide or a pharmaceutically-acceptable salt thereof is loaded into the pores of said silica particles, and wherein the silica particles have:
(a) a mass median aerodynamic diameter that is between about 0.1 μm and about 15 μm (e.g. about 10 μm); and
(b) a geometric standard deviation that is less than about 4.

2. The composition as claimed in claim 1, wherein the loaded silica particles have a mass density that is less than about 0.4 g/cm³.

3. The composition as claimed in claim 1, wherein the mass median aerodynamic diameter is between about 3 μm and about 5 μm.

4. The composition as claimed in claim 1, wherein the geometric standard deviation is between about 1 and about 1.5.

5. The composition as claimed in claim 1, wherein the silica particles have a pore size that is between about 10 nm and about 20 nm.

6. The composition as claimed in claim 1, wherein the silica particles have a pore volume that is between 0.2 and 3 cm³/g.

7. The composition as claimed in claim 1, wherein the silica particles have a surface area that is between about 150 and about 1200 m²/g.

8. The composition as claimed in claim 1, wherein the silica particles are essentially spherical.

9. The composition as claimed in claim 1, wherein up to about 45% of the total weight of the loaded silica particles is N-butyloxycarbonyl-3 -(4-imidazol-1-ylmethylphenyl)-5-iso-butylthiophene-2-sulfonamide, or a pharmaceutically-acceptable salt thereof.

10. The composition as claimed in claim 1, wherein N-butyloxycarbonyl-3-(4-imidazol-1-ylmethylphenyl)-5-iso-butylthiophene-2-sulfonamide or pharmaceutically-acceptable salt thereof is essentially amorphous.

11. The composition as claimed in claim 1, wherein the silica particles consist essentially of a synthetic biodegradable amorphous mesoporous silica.

12. A process for the production of a composition as defined in claim 1, which process comprises providing porous silica particles either:
(a) having a mass median aerodynamic diameter and/or a geometric standard deviation as defined in the relevant preceding claims; or
(b) at least in part not having a mass median aerodynamic diameter and/or a geometric standard deviation as defined in the relevant preceding claims, and thereafter separating silica particles so as to obtain particles having a mass median aerodynamic diameter and/or a geometric standard deviation within those ranges; followed by, in either case,
(c) loading the obtained particles with N-butyloxycarbonyl-3-(4-imidazol-1-ylmethylphenyl)-5-iso-butylthiophene-2-sulfonamide or pharmaceutically-acceptable salt thereof

13. The process as claimed in claim 12, which process further comprises one or more of the steps of:
(i) prior to step (b), calcining the mesoporous silica particles at a temperature of between about 650° C. and about 750° C. in order to provide silica particles; and/or
(ii) after step (b) (and/or after step (i) above), surface modifying said silica particles by chemical reaction thereof with a reagent that provides at least one organic group; and/or
(iii) after step (c), admixing the loaded silica particles with a fatty acid- or a lipid-based surfactant.

14. The process as claimed in claim 12, wherein the silica particles are separated and classified into the desired particle size range using an air classifier or via an elutriation step.

15. The process as claimed in claim 12, wherein the silica particles are loaded with N-butyloxycarbonyl-3-(4-imidazol-1-ylmethylphenyl)-5-iso-butylthiophene-2-sulfonamide or pharmaceutically-acceptable salt thereof using a process of solvent evaporation.

16. The process as claimed in claim 12, wherein the silica particles are manufactured by reacting tetraethyl orthosilicate with a template made of micellar structures.

17. The process as claimed in claim 12, wherein the silica particles are manufactured by a sol-gel method comprising a condensation reaction of an aqueous suspension of silica nanoparticles with a non-miscible organic solution, oil, or liquid polymer, followed by gelation by means of change in pH and/or evaporation of the aqueous phase.

18. A pharmaceutical composition obtained by the process as defined in claim 12.

19. A pharmaceutical formulation comprising a composition as defined in claim 1 in admixture with one or more pharmaceutically-acceptable excipients.

20. The pharmaceutical formulation as claimed in claim 19, wherein the excipient is a hydrocarbon, a fluorocarbon and/or a hydrogen-containing fluorocarbon propellant.

21. The pharmaceutical formulation as claimed in claim 19, which is a dry powder formulation in which the excipient is of larger particle size.

22. A process for the production of a composition, which process comprises admixing the composition as defined in claim 1, with the one or more pharmaceutically-acceptable excipients.

23-24. (canceled)

25. A method of treatment of an interstitial lung disease, which method comprises the administration of a pharmacologically-effective amount of a composition as defined in claim 1, to a patient in need of such treatment.

26. The method of treatment as defined in claim 25, wherein the interstitial lung disease is idiopathic pulmonary fibrosis.

27. The method of treatment as defined in claim 25, wherein the interstitial lung disease is sarcoidosis.

28. The method of treatment as defined in claim 25, wherein the composition is administered by the pulmonary route.
